(19) United States
(12) Patent Application Publication
(10) Pub. No.: US 2020/0408787 A1
(43) Pub. Date: Dec. 31, 2020

(54) STABILIZED QUALITY CONTROL MATERIALS FOR RED BLOOD CELLS FOR DIAGNOSTIC TESTS

(71) Applicant: NEW DIAGNOSTIC SERVICES LIMITED, Somerset (GB)

(72) Inventor: Craig HALLETT, Somerset (GB)

(21) Appl. No.: 16/975,251

(22) PCT Filed: Feb. 11, 2019
(86) PCT No.: PCT/GB2019/050358

§ 371 Date: Aug. 24, 2020

(30) Foreign Application Priority Data
Feb. 26, 2018 (GB) 1803065.0

(51) Int. Cl. G01N 33/96; G01N 33/72

(52) U.S. Cl. CPC G01N 33/96; G01N 33/72

(57) ABSTRACT

A quality control material, its method of preparation and it use as a control composition for diagnostic tests are described. The quality control material comprises: non-fixed viable red blood cells; and a support medium comprising: abutter; a polyol; adenine and fructose.

### FIELD OF THE INVENTION

The invention relates to a quality control material for red blood cells, its method of preparation and its use as a quality control material for diagnostic tests. In particular, it relates to a quality control material that closely mimics a whole blood sample but which has long term stability and is suitable for quality control purposes.

### BACKGROUND

Many blood component containing control materials are known. Where applied to serum analyses, freezing such samples may bean adequate method of preservation. For clinical tests which measure the concentration/amount of a substance within a red cell, such as glycated haemoglobin (GHB) or haemoglobin A1c (HbA1c), a control material containing red cells is required to be commutable with patient samples used. For example, in point of care or near patient testing devices for glucose or lipids, freezing of samples is not appropriate as it will cause lysis of the red cells making it unsuitable for use with some analysers e.g. Affinion AS100. Use of serum/plasma based control material may influence the results obtained dependent on the device used.

In some situations, pooled patient/normal samples are an accepted means of producing an unassayed quality control material. Specifically relating to HbA1c measurement on the Affinion AS100 analyser, the manufacturer claims that whole blood samples stored at 4° C. are suitable for analysis for up to 10 days after collection, although there is a degree of variability within individuals which limits the usefulness of unpreserved blood as a control material. Preserved liquid or lyophilised controls are available for these analyses, but have the disadvantage that the GH B/HbA1c is not within cells and thus cannot provide assurance regarding the necessary lysis step of the analysis.

Cellular controls are available but rely on resuspension in alternative media after fixation of the red blood cells with e.g. glutaraldehyde. For example, U.S. Pat. No. 7,361,513 discloses a cellular control for glycated haemoglobin HbA 1c that uses glutaraldehyde (25% in a cell wash dilutent) to fix the red blood cells. US2014/0134597 discloses a cellular haemoglobin HbA1c quality control that requires dialyzing red blood cells against a hypotonic solution to cause permeabilization of the cell membranes, infusing the red blood cells with HbA1c and then de-permeabilzation of the cell membranes.

US2014/0134597 also discloses subsequently fixing the red blood cells. Such fixing steps may compromise the commutability of the samples to a significant degree.

In addition, many of the known red blood cell controls require the presence of glucose. However, this can be problematic because over ti me the glucose in such controls produces HbA1c by non-specific chemical reactions. This changes the composition of the control over time and is particularly problematic where it is desired to measure the HbA1c level as is commonly used in the diagnosis, monitoring and control of diabetes.

Hence, there is a need to produce a control material which more closely resembles clinical samples, which has long term stability and which minimizes any degradation of HbA1c. Ideally, such control materials should be free of interfering substances and have similar viscosity, pH and osmolality to clinical blood samples.

The present invention seeks to alleviate the problems in the prior art. In particular, this invention preserves the red blood cells with minimal intervention, so as to produce a control material where the red cells are as near to their native state after collection as is possible, but extending the time where they can be used as an unassayed control with no degradation of HbA1c/glycated haemoglobin within the red cell. These quality control materials contain red blood cells that remain viable for prolonged periods, both in terms of intracellular components, i.e. HbA1c and as the base matrix for a whole blood internal quality control or external quality assurance material containing components of interest in clinical pathology (both human and animal).

### SUMMARY

In a first aspect, the present invention provides a quality control material comprising:
non-fixed viable red blood cells; and
a support medium comprising:
   a buffer;
   a polyol ;
   adenine; and
   fructose.

In a further aspect, the present invention provides the use of a quality control material according to any one of claims as a diagnostic test control.

In a further aspect, the present invention provides the use of quality control material as a diagnostic test control for a di agnostic test of glycated haemoglobin or haemoglobin A1c.

In a further aspect, the present invention provides a method for preparing a quality control material as described herein, comprising the steps of:
(i) selecting a sample of red blood cells with at least one desired feature from suitable subjects;
(ii) processing the sample to remove white blood cells;
(iii) processing the sample to remove the majority of the plasma;
(iv) optionally washing the sample of step (iii);
(v) optionally re-suspending the sample of step (iv) in plasma;
(vi) admixing the sample of step (v) with a support medium to produce the quality control material as described herein.

In a further aspect, the present invention provides a method for determining the accuracy and reproducibility of the operation of an analytical instrument capable of measuring an analyte of interest comprising:
(a) providing a quality control material as described herein where reference values have been determined;
(b) determining the level of the analyte of interest in the quality control material of (a); and
(c) comparing the level of the analyte of interest obtained in (b) with the known reference values; wherein said comparing indicates the accuracy and reproducibility of the operation of the analytical instrument.

In a further aspect, the present invention provides a quality control material comprising:
non-fixed vi able red blood cells; and
a support medium comprising:
   a buffer;
   a polyol;
   adenine; and
   an energy source.

In a further aspect, the present invention provides a quality control material comprising:
non-fixed viable red blood cells; and
a support medium comprising:
   a buffer;
   a polyol;
   adenine;
   an energy source (for example, fructose); and
   an antimicrobial agent.

Further particular and preferred aspects are set out in the accompanying independent and dependent claims. Features of the dependent claims may be combined with features of the independent claims as appropriate, and in combinations other than those explicitly set out in the claims.

As used herein the term “comprising” means “including at least in part of” and is meant to be inclusive or open ended. When interpreting each statement in this specification that includes the term “comprising”, features, elements and/or steps other than that or those prefaced by the term may also be present. Related terms such as “comprise” and “comprises” are to be interpreted in the same manner.

The term “consisting essentially of” limits the scope of a claim to the specified materials or steps “and those that do not materially affect the basic and novel characteristic(s)” of the claimed invention. When the phrase “consisting essentially of” appears in a clause of the body of a claim, rather than immediately following the preamble, it limits only the element set forth in that clause.

The term “consisting of” excludes any element, step, or ingredient not specified in the claim; “consisting of” defined as “closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole. It should be understood that while various embodiments in the specification are presented using “comprising” language, under various circumstances, a related embodiment is also described using “consisting essentially of” or “consisting of” language.

As used herein the term “ester” suitably refers to an alkyl ester of carboxylic acid. Suitably, the ester is a C₁₋₇ alkyl ester. That is an alkyl ester containing an alkyl group that is a straight chain or a branched saturated hydrocarbon group, generally having from 1 to 7 carbon atoms. Suitably the alkyl ester is selected from methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl and n-heptyl esters.

As used herein the term “non-fixed viable red blood cells” means red blood cells that have not undergone chemical fixation, such as by treating with glutaraldehyde, which can significantly modify the red blood cells membranes and intracellular proteins. As used herein the term “non-fixed viable red blood cells” also means red blood cells that survive and preserve their membrane integrity (minimising haemolysis) and maintaining haemoglobin. Test may be performed to check that the supernatant haemoglobin does not indicate mass lysis; and/or to check the cell counts on storage; and/or to check any deterioration of haemoglobin on storage by checking the haemoglobin on chromatography.

“Optionally components” refers to components that may not be present, hence, there may be 0, 1 or more optional components present. Suitably, there are 0, 1, 2, 3, 4, 5, 6, 7, 8, 9 or 10 optional components.

As used herein the term “polyol” means a compound comprising two or more hydroxyl groups but no other functional groups. Suitably the polyol comprises 2, 3, 4, 5 or 6 hydroxyl groups.

As used herein the term “quality control material” means a material that is suitable for use as an internal quality control, or as an external quality assurance material, or for proficiency testing purposes.

### Non-Fixed Red Blood Cells

Suitably, the non-fixed red blood cells are suspended in plasma.

Suitably, the volume/ volume ratio of the red blood cells to plasma is from 1:1 to 10:1; more suitably, from 1:1 to 9:1, from 1:1 to 8:1, from 1:1 to 7:1, from 1:1 to 6:1, and from 1:1 to 5:1.

Suitably, the volume/volume ratio of the red blood cells to plasma is from 2:1 to 10:1; more suitably, from 2:1 to 9:1, from 2:1 to 8:1, from 2:1 to 7:1, from 2:1 to 6:1, and from 2:1 to 5:1.

Suitably, the volume/volume ratio of the red blood cells to plasma is from 3:1 to 10:1; more suitably, from 3:1 to 9:1, from 3:1 to 8:1, from 3:1 to 7:1, from 3:1 to 6:1, and from 3:1 to 5:1.

In some embodiments, suitably, the volume/volume ratio of the red blood cells to plasma is about 4:1.

Suitably, the red blood cells suspended in plasma have a haematocrit of between 65% and 90%; more suitably, a haematocrit of between 67% and 90%.

### Support Medium

Suitably, the support medium comprises a buffer; polyol; adenine and an energy source for the red blood cells (for example, fructose); and

one or more optional components selected from an antimicrobial agent, an antioxidant, protein, a glucose transport 1 inhibitor, additives and mixtures thereof.

Suitably, the support medium comprises a buffer; polyol; an energy source for the red blood cells (for example, fructose); an antimicrobial agent; adenine; an antioxidant; protein; and a glucose transport 1 inhibitor.

### Adenine

Adenine is a nucleobase purine derivative with a chemical formula C₅H₅N₅. Adenine improves production of adenosinetriphosphate (ATP) by the stored red blood cells.

Suitably, the support medium comprises from 0.1-10.0 mM concentration of adenine.

Suitably, the support medium comprises from 0.5-8.0 mM concentration of adenine.

Suitably, the support medium comprises from 0.7-5.0 mM concentration of adenine.

### Antimicrobial Agent

Suitably, the quality control material further comprises an antimicrobial agent.

Suitably, the support medium further comprises an antimicrobial agent.

Suitably, the antimicrobial agent comprises an agent selected from an antibiotic agent, an antifungal agent and mixtures thereof. Suitably, the antimicrobial agent is an antibiotic agent, an antifungal agent or a mixture thereof.

Suitably, the antimicrobial agent is an antibiotic agent.

Suitably, the antimicrobial agent is selected from gentamicin, neomycin sulfate, chloramphenicol, esters of para-hydroxybenzoic acid, a sorbate salt and mixtures thereof.

Suitably, the antimicrobial agent is selected from gentamicin, neomycin sulfate, chloramphenicol, and mixtures thereof.

More suitably, the antimicrobial agent is gentamicin.

Suitably, the support medium comprises from 1-30 mg/L of an antimicrobial agent; suitably, from 5-25 mg/L; or from 10-20 mg/L of an antimicrobial agent.

### An Antioxidant

Suitably, the reference control material further comprises an antioxidant.

Suitably, the support medium further comprises an antioxidant.

Suitably, the antioxidant comprises an antioxidant selected from tocopherol acetate, N-acetyl cysteine, ascorbic acid, ascorbic acid polypeptide, ascorbyl dipalmitate, ascorbyl methylsilanol pectinate, ascorbyl palmitate, ascorbyl stearate, betalains (betanin), betaxanthine (e.g., indicaxanthine) BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, methyl paraben, esters of para-hydroxybenzoic acid, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, vitamin D, quinic acid, chlorogenic acid, glutathione, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, tris(nonylphenyl)phosphite and mixtures thereof.

Suitably, the antioxidant comprises an antioxidant selected from tocopherol acetate, N-acetyl cysteine, ascorbic acid, methyl paraben, esters of para-hydroxybenzoic acid, and mixtures thereof.

More suitably, the antioxidant comprises an antioxidant selected from tocopherol acetate, N-acetyl cysteine, ascorbic acid, and mixtures thereof.

Suitably, the support medium comprises from 0.10-1.50 mM concentration of an antioxidant. Suitably, the support medium comprises from 0.25-1.00 mM concentration of an antioxidant.

More suitably, the antioxidant comprises N-acetyl cysteine. Suitably, the support medium comprises from 0.09-1.00 mM concentration of N-acetyl cysteine. Suitably, the support medium comprises from 0.25 to 0.75 mM concentration of N-acetyl cysteine.

More suitably, the antioxidant comprises ascorbic acid. Suitably, the support medium comprises from 0.01-0.50 mM concentration of ascorbic acid. Suitably, the support medium comprises from 0.10-0.30 mM concentration of ascorbic acid.

More suitably, the antioxidant comprises N-acetyl cysteine and ascorbic acid. The use of the antioxidant N-Acetyl cysteine (NAc) leads to improved glutathione (GSH) accumulation in stored red cells protecting from oxidative damage to haemoglobin (Pallottta, V. et al., Blood Transfus, 2014, vol. 12, pages 376-387). Ascorbic acid has an effect in conjunction with NAc. Ascorbic acid competes with glucose for transport into red cell, this can provide improved stability if an energy source such a fructose is used, as potentially there will be no reduction in ATP production due to fructose not competing with it for transport.

### Buffer

Suitably, the buffer comprises components selected from N-(2-Acetamido)-2-aminoethanesulfonic acid (ACES); 1,4-Piperazinediethanesulfonic acid (PIPES); *-hydroxy-4-morpholinepropanesulfonic acid (MOPSO); 3-Bis[tris(hydroxymethyl)-methylamino]propane; N,N-bis(2-hydroxyethyl)-2-aminoethanesulfonic acid (BES); 3-(N-morpholino)propanesulfonic acid (MOPS); 2-[(2-hydroxy-1,1-bis(hydroxymethyl)-ethyl)amino]ethanesulfonic acid (TES); 4-(2-hydroxyethyl)piperazine-1-ethanesulfonic acid (HEPES); 3-(N,N-bis[2-hydroxyethyl]amino)-2-hydroxypropanesulfonic acid (DIPSO); 4-(N-morpholino)butanesulfonic acid (MOBS); 2-hydroxy-3-[tris-(hydroxymethyl)methylamino]-1-propanesulfonic acid (TAPSO); 2-amino-2-(hydroxymethyl)-1,3-propanediol (Trizma); 4-(2-hydroxyethyl)piperazine-1-(2-hydroxypropanesulfonic acid) (HEPPSO); piperazine-1,4-bis(2-hydroxypropane-sulfonic acid) (POPSO); triethanolamine; 4-(2-hydroxyethyl)-1-piperazinepropane-sulfonic acid; 4-(2-Hydroxyethyl)piperazine-1-propanesulfonic acid (EPPS); N-[tris(hydroxymethyl)methyl]glycine(tricine); diglycine (Gly-Gly); N,N-bis(2-hydroxyethyl)glycine (buicine); N-(2-hydroxyethyl)piperazine-N-(4-butanesulfonic acid (HEPBS); N-[tris(hydroxymethyl)methyl]-3-aminopropanesulfonic acid (TAPS); 2-amino-2-methyl-1,3-propanediol (AMPD); citric acid or a salt thereof; phosphate salts and mixtures thereof.

Suitably, the buffer comprises components selected from citric acid or a salt thereof; phosphate salts and mixtures thereof. Suitably, the buffer comprises components selected from citric acid or a salt thereof; alkali metal phosphate salts and mixtures thereof.

Suitably, the buffer comprises components selected from citric acid; sodium citrate; potassium citrate; monosodium phosphate; disodium phosphate; monopotassium phosphate; dipotassium phosphate and mixtures thereof.

More suitably, the buffer comprises potassium citrate, monopotassium phosphate and dipotassium phosphate.

Suitably, the support medium comprises from 5-25 mM concentration of citric acid or a salt thereof. Suitably, the support medium comprises from 10-20 mM concentration of citric acid or a salt thereof.

Suitably, the support medium comprises from 10-70 mM concentration of phosphate salts. Suitably, the support medium comprises from 20-60 mM concentration of phosphate salts. Suitably, the support medium comprises from 30-50 mM concentration of phosphate salts.

More suitably, the support medium comprises from 20-50 mM concentration of disodium phosphate or dipotassium phosphate. More suitably, the support medium comprises from 30-40 mM concentration of disodium phosphate or dipotassium phosphate.

More suitably, the support medium comprises from 1-10 mM concentration of monosodium phosphate or monopotassium phosphate. More suitably, the support medium comprises from 2-8 mM concentration of monosodium phosphate or monopotassium phosphate.

Suitably, the support medium is buffered to pH 7.4 to 7.8.

### Energy Source

In some aspects, the quality control material comprises an energy source selected from dextrose/glucose or fructose. Suitably, the support medium comprises an energy source for the red blood cells selected from a pentose (e.g. arabinose, lyxose, ribose, xylose, ribulose and xylulose) dextrose/glucose or fructose.

Most suitably, the energy source i s fructose.

Suitably, the support material comprises from 20-200 mM concentration of the energy source. Suitably, the support material comprises from 50-150 mM concentration of the energy source. Suitably, the support material comprises from 75-125 mM concentration of the energy source.

### Polyol

The polyol is a free radical scavenger.

Suitably, the polyol is selected from a sugar alcohol, glycerol and mixtures thereof.

More suitably, the polyol is selected from mannitol, sorbitol, xylitol, glycerol and mixtures thereof.

Most suitably, the polyol is mannitol. Mannitol is a sugar alcohol with the molecular formula C₆H₁₄O₆.

Suitably, the support material comprises from 10-100 mM concentration of polyol. Suitably, the support material comprises from 25-75 mM concentration of polyol. More suitably, the support material comprises from 45-65 mM concentration of polyol.

### Protein

Suitably, the reference control material further comprises a protein.

Suitably, the support medium further comprises a protein.

More suitably, the protein comprises a protein selected from bovine serum albumin, succinylated gelatine and mixtures thereof.

Suitably, the support medium comprises from 5 to 90 g/L of a protein. More suitably, the support medium comprises from 10 to 70 g/L of a protein; more suitably, from 20-60 g/L of a protein; more suitably, from 30 to 50 g/L of a protein.

The protein may (a) increase viscosity of medium to mimic true plasma, and/or (b) provide additional buffering capacity.

### Glucose Transport 1 (GLUT1) Inhibitors

Suitably, the composition further comprises a glucose transport 1 inhibitor.

Suitably, the support medium further comprises a glucose transport 1 inhibitor.

Suitably, the glucose transport 1 inhibitor comprises a glucose transport 1 inhibitor selected from genistein, fasentin and mixtures thereof.

A GLUT1 inhibitor may be added where material will be used for glucose control material so as to block utilisation of glucose by red cells when present in suspension medium. It does not inhibit the GLUT transporter responsible for the uptake of fructose into the red cell because fructose, which may be provided as energy source, uses an alternate GLUT transport protein.

Suitably, the support medium comprises from 10-500 μM concentration of a glucose transport 1 inhibitor. Suitably, the support medium comprises from 50-200 μM concentration of a glucose transport 1 inhibitor.

### Additives

Suitably, the composition further comprises one or more additives. Suitably, the support medium comprises one or more additives.

Suitably, the one or more additives are selected from a stabilizer, an HbA1c enriched haemoglobin solution, a pH adjuster, a protease inhibitor, an analyte of interest and mixtures thereof.

Suitably, the one or more additives comprise a stabilizer. Suitably, the stabilizer is selected from magnesium gluconate, EDTA (ethylene-diamine tetraacetic acid) PEG (polyethylene glycol) and mixtures thereof.

Such, the one or more additives comprise an HbA1c enriched haemoglobin solution. HbA1c enrichment can be achieved by ion-exchange chromatography, boronic acid affinity chromatography or a combination of both. Hb in supernatant is stable in support medium with no evidence of metHb formation after 63 days.

Suitably, the one or more additives comprise a pH adjuster. Suitably, the pH adjuster is an acid or abase. Suitably, the pH adjuster is selected from HCl, sodium hydroxide, potassium hydroxide and mixtures thereof.

Suitably, the one or more additives comprise a protease inhibitor. Suitably, the protease inhibitor is soybean trypsin inhibitor.

Suitably, the one or more additives may comprise analytes of interest.

The suspended red cell solutions can be utilised a base material for creating “whole blood” control materials by the addition of other analytes of interest.

### Analytes of Interest

The present disclosure describes quality control material that is useful for the quality control testing and calibration of various analytes of interest in the context of clinical pathology (human and veterinary pathology).

In some aspects, the present disclosure provides quality control material useful to the quality control testing and calibration of various analytes of interest in the context of diabetes management including blood cell analytes (primarily, glycated hemoglobin), plasma analytes (e.g., glucose and ketone).

Embodiments may be utilized to provide such quality control material and methods suitable for the quality control testing and calibration in the analysis of the ratio of glycated hemoglobin, hemoglobin A1c, and additional variants including, e.g., HbA1, HbA2, HbC, HbF, and HbS.

Additionally, embodiments of this disclosure may be utilized to provide comprehensive quality control material and methods for their preparation and use suitable for the quality control testing and calibration in the analysis of multiple analytes of interest in the context of clinical pathology including blood cell analytes (primarily, glycated hemoglobin) and plasma analytes (e.g., glucose and ketone).

Accordingly, the present disclosure provides quality control material and methods useful for the quality control testing and calibration for the detection and monitoring of analytes of interest in clinical pathology. Suitably, the analytes of interest are those medically involved in the diagnosis and management of diabetes and diabetes related conditions.

In some embodiments analytes of interest include those of relevance in monitoring the concentration of a given drug administered to a patient including but not limited to drugs routinely administered in the management of diabetes and diabetes related conditions (see generally Goodman and Gil man's The Pharmacological Basis of Therapeutics, 13th Ed., McGraw Hill Companies Inc., New York (2017)) e.g., insulin. However, quality control material may encompass a variety of analytes of interest which may not be necessarily related to diabetes and/or related conditions.

Thus, as utilized herein the term “analyte” denotes any material of interest. Analytes of interest may include, but are not limited to, glucose, cholesterol (high density, low density and total cholesterol), triglycerides, fructosamine, amino acids, electrolytes (Na⁺, K⁺, and Cl⁻), urea, uric acid, lactate, ketones, ketone bodies (acetoacetate and 3-hydroxybutyrate), hemoglobin, glycosylated hemoglobin, albumin, creatine, creatinine, metabolites related to disease, drugs and drug metabolites, pesticides, haptens, steroid hormones, vitamins, trace elements (e.g. cobalt, copper, fluorine, iodine, iron, manganese and zinc) and antigens (e.g., components of peptides, proteins, polysaccharides, nucleic acids, bacteria, viruses, chromosomes, genes, mitochondria, nuclei, cell membranes, and the like), and antibodies.

Drugs of interest as analytes include alkaloids, steroids, lactams, aminoalkylbenzenes, benzheterocyclics, purines, vitamins, prostaglandins, antibiotics, nucleosides, nucleotides, aminoglycosides, cannabinol and tetrahydrocannabinol. Alkaloids include morphine alkaloids (e.g., morphine, codeine, heroin, and dextromethorphan), cocaine alkaloids (e.g., cocaine and benzoyl ecgonine), ergot alkaloids (e.g., diethylamide of lysergic acid), steroid alkaloids, iminazoyl alkaloids, quinazoline alkaloids, isoquinoline alkaloids, quinoline alkaloids (e.g., quinine and quinidine), diterpene alkaloids, and their derivatives and metabolites. Steroid analytes include estrogens, estogens, progestogens, androgens, andreocortical steroids, bile acids, cardiotonic glycosides and aglycones (e.g., digoxin and digoxigenin), saponins and sapogenins, and their derivatives and metabolites. Steroid mimetic substances, such as diethylstilbestrol, are also drug analytes of interest. Lactam analytes include barbituates (e.g., phenobarbital and secobarbital), diphenylhydantonin, primidone, ethosuximide, and their derivatives and metabolites. Aminoalkylbenzenes analytes include amphetamines, catecholamines (e.g., ephedrine, L-dopa, epinephrine, narceine, papaverine), and their derivatives and metabolites. Benzheterocyclic analytes include drugs that have an azepine, diazepine or phenothiazine heterocyclic ring compounds, such as oxazepam, chlorpromazine, tegretol, imipramine, and their derivatives and metabolites. Purineanalytes include theophylline, caffeine, and their derivatives and metabolites. Vitamin analytes of interest include A, B, B12, C, D, K, folic acid and thiamine. Antibiotic analytes include penicillin, chloromycetin, actinomycetin, tetracycline, teramycin, and their derivatives and metabolites. Nucleoside and nucleotide analytes include adenosine triphosphate (ATP), nicotinamide adenine dinucleotide (NAD), flavin mononucleotide (FM N), adenosine, guanosine, thymidine, and cytidine, and their derivatives and metabolites. Other drug analytes of interest include methadone, meprobamate, serotonin, meperidine, amitriptyline, nortriptyline, lidocaine, procaineamide, acetylprocaineamide, propanolol, griseofulvin, valproic acid, butyrophenones, antihistamines, anticholinergic drugs (e.g., atropine), and their derivatives and metabolites. Metabolites related to disease states which may be analytes of interest include spermine, galactose, phenylpyruvic acid, and porphyrin. Pesticides of interest include polyhalogenated biphenyls, phosphate esters, thiophosphates, carbamates, polyhalogenated sulfonamides, and their derivatives and metabolites.

In exemplary embodiments, the analyte of interest can be glucose. Generally, glucose may be present in a solution of the disclosure in a concentration range of between about 10 mg/dL to about 500 mg/dL.

### Quality Control Material

Suitably, the red blood cells are suspended in plasma and the volume/volume ratio of red blood cells in plasma : support medium is from 2:1 to 1:5; more suitably, the ratio is from 2:1 to 1:4, more suitably, the ratio is from 2:1 to 1:3, more suitably, the ratio is from 2:1 to 1:2.

In some embodiments, suitably, the red blood cells are suspended in plasma and the volume/volume ratio of red blood cells in plasma : support medium is about 1:1.

Suitably, the quality control material has a pH from 7.4 to 7.8 at 20° C.

In some aspects, suitably, the quality control material has a osmolality from 250-350 mOsmo/kg.

### Method of Preparation

Suitably, the sample of red blood cells in step (i) are pooled samples from suitable donors.

Suitably, the pooled samples from suitable donors are collected in an anticoagulant.

In some aspects, suitably the suitable donors are from normal healthy individuals. In other aspects, the suitable donors are patients. Suitably, the patients are diabetic patients.

Suitably, the processing of the sample to remove the white blood cells in step (ii) comprises centrifugation followed by removal of the buffy coat; or in-line filtration. More suitably, the processing in step (ii) comprises in-line filtration. Such in-line filtration may be carried out using in-line leukoreduction filter such as that of Hemonetics.

Suitably, step (iii) processing the sample to remove the majority of the plasma comprises either centrifugation or plasmapheresis. Suitably, from 50-95% of the original plasma is removed. More suitably, from 65-95%, from 70-95%, from 75-95%, from 80-95%, or from 85-95% of the original plasma is removed.

In some aspects in step (iii) at least 50%, at least 65%, at least 70%, at least 75%, at least 80%, or at least 85% of the original plasma is removed.

Suitably, the method comprises step (iv) washing the sample of step (iii). Suitably, step (iv) comprises washing the sample of (iii) in awash solution containing a buffer, mannitol and an energy source for the red blood cells. Suitably, the wash solution further comprises adenine.

Suitably, the method comprises step (v) re-suspending the sample of step (iv) in plasma. Suitably, in step (v) the washed sample from step (iv) is re-suspended in plasma. Suitably, the volume/ volume ratio of the washed sample of red blood cells to plasma is from 1:1 to 10:1; more suitably, from 1:1 to 9:1, from 1:1 to 8:1, from 1:1 to 7:1, from 1:1 to 6:1, and from 1:1 to 5:1. In some embodiments, suitably, the volume/volume ratio of the washed sample of red blood cells to plasma is from 2:1 to 10:1; more suitably, from 2:1 to 9:1, from 2:1 to 8:1, from 2:1 to 7:1, from 2:1 to 6:1, and from 2:1 to 5:1. In some embodiments, suitably, the volume/volume ratio of the red blood cells to plasma is from 3:1 to 10:1; more suitably, from 3:1 to 9:1, from 3:1 to 8:1, from 3:1 to 7:1, from 3:1 to 6:1, and from 3:1 to 5:1.

In some embodiments, suitably, the volume/volume ratio of the washed sample of red blood cells to plasma is about 4:1.

Suitably, in step (vi) the sample of step (v) is admixed with a support medium to produce the quality control material as described herein. Suitably, the sample of step (v) comprising red blood cells suspended in plasma is admixed with a support medium in a volume/volume ratio of red blood cells in plasma: support medium is from 2:1 to 1:5; more suitably, the ratio is from 2:1 to 1:4, more suitably, the ratio is from 2:1 to 1:3, more suitably, the ratio is from 2:1 to 1:2. In some embodiments, suitably, the ratio is about 1:1.

### Uses

Suitably, the quality control material is used as a diagnostic test control.

Suitably, the quality control material is used as a diagnostic test control for a diagnostic test of glycated haemoglobin or haemoglobin A1c.

### Other Forms

Unless otherwise specified, included in the above are the well known ionic, salt, solvate, and protected forms of these substituents. For example, a reference to carboxylic acid (—COOH) also includes the anionic (carboxylate) form ('COO^{—}), a salt or solvate thereof, as well as conventional protected forms. Similarly, a reference to an amino group includes the protonated form (—N+HR¹R²), a salt or solvate of the amino group, for example, a hydrochloride salt, as well as conventional protected forms of an amino group. Similarly, a reference to a hydroxyl group also includes the anionic form (—O^{—}), a salt or solvate thereof, as well as conventional protected forms.

### Isomers, Salts and Solvates

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r- forms; endo- and exo-forms; R-, S-, and meso-forms; D- and L-forms; d- and I- forms; (+) and (−) forms; keto-, enol-, and enolate-forms; syn- and anti-forms; synclinal- and anticlinal-forms; alpha- and beta-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH .

A reference to a class of structures may well include structurally isomeric forms falling within that d as (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not apply to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro. In some cases, the quality control material may comprise components that can exist as tautomers.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof.

Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

Component compounds of the composition, which include compounds specifically named above, may form complexes, salts, solvates and hydrates, in particular, may form pharmaceutically acceptable complexes, salts, solvates and hydrates. These salts include acid addition salts (including di-acids) and base salts; in particular nontoxic acid addition salts (including di-acids) and base salts.

If the component compound is cationic, or has a functional group which may be cationic (e.g. —NH₂ may be —NH₃⁺), then an acid addition salt may be formed with a suitable anion. Examples of suitable inorganic anions include, but are not limited to, those derived from the following inorganic acids hydrochloric acid, nitric acid, nitrous acid, phosphoric acid, sulfuric acid, sulphurous acid, hydrobromic acid, hydroiodic acid, hydrofluoric acid, phosphoric acid and phosphorous acids. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydriodide/iodide, isethionate, lactate, malate, maleate, malonate, mesylate, methylsulfonate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate and xinofoate salts.

For example, if the component compound is anionic, or has a functional group which may be anionic (e.g. —COOH may be —COO^{—}, or —SO₂H may be —SO₂^{—}, then a base salt may be formed with a suitable cation. Examples of suitable inorganic cations include, but are not limited to, metal cations, such as an alkali or alkaline earth metal cation, ammonium and substituted ammonium cations, as well as amines. Examples of suitable metal cations include sodium (Na⁺) potassium (K⁺), magnesium (Mg²⁺), calcium (Ca²⁺), zinc (Zn²⁺), and aluminum (Al³⁺). Examples of suitable organic cations include, but are not limited to, ammonium ion (i.e. NH4⁺) and substituted ammonium ions (e.g. NH₃R⁺, NH₂R₂^{+, NHR}₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such aslysine and arginine. An example of a common quaternary ammonium ion is N(CH₃)₄⁺. Examples of suitable amines include arginine, N,N′-dibenzylethylene-diamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine.

For a discussion of useful acid addition and base salts, see S. M. Berge et al., J. Pharm. Sci. (1977) 66:1-19; see also Stahl and Wermuth, Handbook of Pharmaceutical Salts: Properties, Selection, and Use (2011)

Salts, such as pharmaceutically acceptable salts, may be prepared using various methods. For example, one may react a component compound with an appropriate acid or base to give the desired salt. One may also react a precursor of the component compound with an acid or base to remove an acid- or base-labile protecting group or to open a lactone or lactam group of the precursor. Additionally, one may convert a salt of the component compound to another salt through treatment with an appropriate acid or base or through contact with an ion exchange resin. Following reaction, one may then isolate the salt by filtration if it precipitates from solution, or by evaporation to recover the salt. The degree of ionization of the salt may vary from completely ionized to almost non-ionized.

It may be convenient or desirable to prepare, purify, and/or handle a corresponding solvate of the active compound. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, acetone-d6, DMSO-d6).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will typically be observed.

These component compounds may be isolated in solid form, for example, by lyophilisation.

Further particular and preferred aspects are set out in the accompanying independent and dependent claims. Features of the dependent claims may be combined with features of the independent claims as appropriate, and in combinations other than those explicitly set out in the claims.

### BRIEF DESCRIPTION OF THE DRAWINGS

Embodiments of the present invention will now be described further, with reference to the accompanying drawings, in which:

FIG. 1 shows an open stability study using measurement of HbA1c using the Tosoh G7 platform where samples A & B contained glucose as the energy source and samples C & D contained fructose as the energy source.

FIG. 2 shows an open stability study using measurement of HbA1c using the Affinion AS100 platform where samples A & B contained glucose as the energy source and samples C & D contained fructose as the energy source.

FIG. 3 shows an open stability study using measurement of HbA1c using the BioRad B100 platform where samples A & B contained glucose as the energy source and samples C & D contained fructose as the energy source.

FIG. 4 shows a long-term stability study using measurement of HbA1c using the Tosoh G7 platform where samples A & B contained glucose as the energy source and samples C & D contained fructose as the energy source.

FIG. 5 shows a haemolysis study where samples A & B contained glucose as the energy source and samples C & D contained fructose as the energy source.

### DESCRIPTION OF THE EMBODIMENTS

### EXAMPLE 1

In essence, blood from suitable donors is collected into an appropriate anticoagulant such as ACD, CPDA1, CPD-SAGM and pooled. The red blood cells are separated from the white blood cells by a suitable process, either by centrifugation followed by removal of the buffy coat, or preferably through in-line filtration e.g. using Hemonetics in-line leukoreduction filter. Removal of white cells may occur prior to pooling of samples, in particular, if in-line filtration is used.

After removal of the white cells, the majority of the plasma is removed to leave the red cells suspended in between 5 and 50% of the original plasma volume to give a haematocrit of between 67% and 90%

The plasma may be reduced through removal after centrifugation, or by passage through an appropriate plasmapheresis filter, either before or after addition of an appropriate volume of preservative support medium.

More specifically, the samples were prepared using the following procedure

1. Standard blood collection in CPD/CPDA followed by removal of white cells by in line filter.

2. Centrifugation/plasmapheresis to remove majority of plasma (reserved)

3. Cells washed in wash solution (see below)×1

4. Washed cells (4 volumes) resuspended in:

5. 1 Volume plasma+5 volumes of support medium (see below)

6. Final adjustment to give desi red haematocrit/haemoglobin concentration by adding appropriate amount of support medium

### Wash Solution:

| | | |
|---|---|---|
| Fructose or glucose | 110 | mM |
| Mannitol | 55 | mM |
| K₂HPO₄ | 34.6 | mM |
| KH₂PO₄ | 5.36 | mM |
| Adenine | 2 | mM |
| Potassium Citrate | 17.9 | mM |

### Support Medium:

| | | |
|---|---|---|
| Fructose or glucose | 110 | mM |
| Mannitol | 55 | mM |
| K₂HPO₄ | 34.6 | mM |
| KH₂PO₄ | 5.36 | mM |
| Adenine | 2 | mM |
| Potassium Citrate | 17.9 | mM |
| Bovine serum albumin | 40 | g/l |
| N-Acetylcysteine | 0.5 | mM |
| Ascorbic acid | 0.23 | mM |

Hence, the support medium in this example contained all of the components of the corresponding wash solution with the addition of further components

Other components of interest can be added to the support medium in order to produce the desired control material.

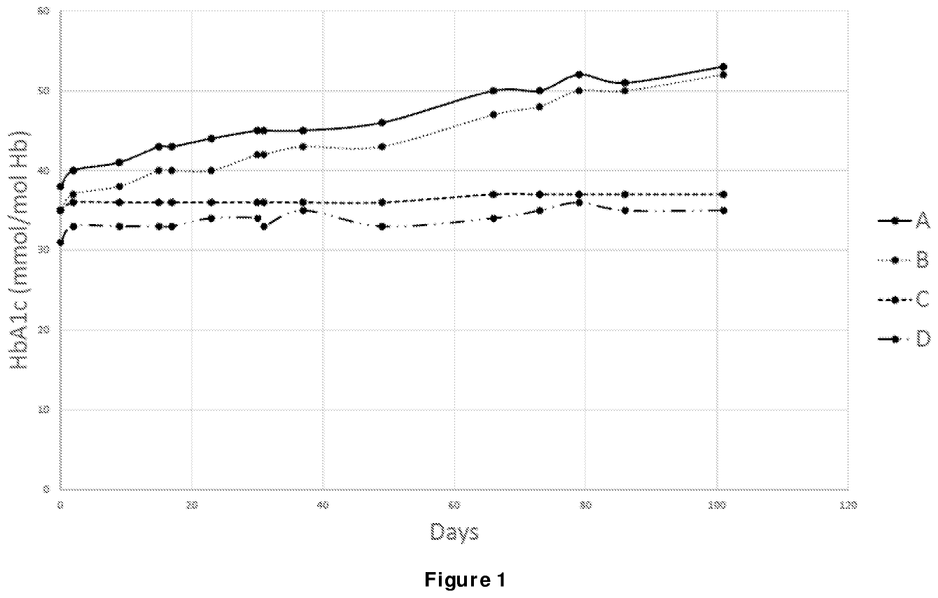

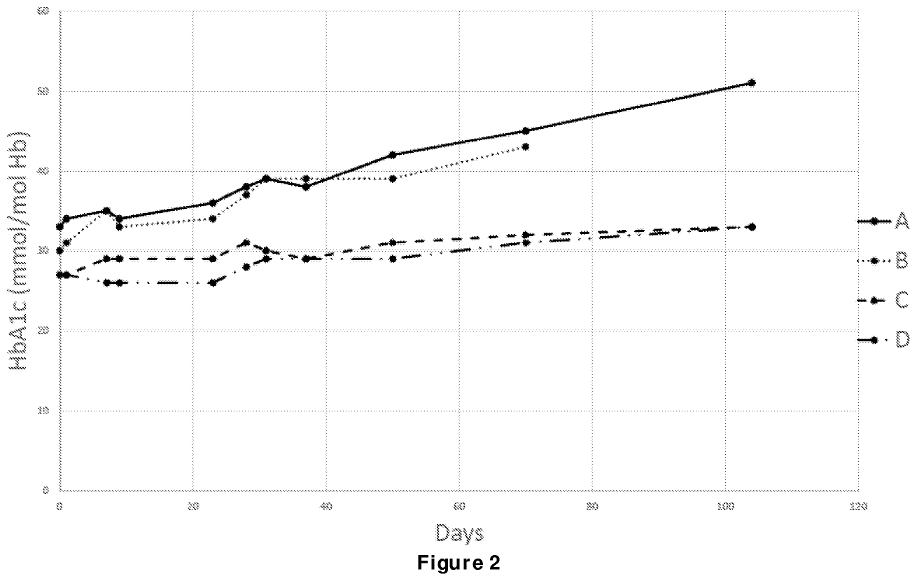

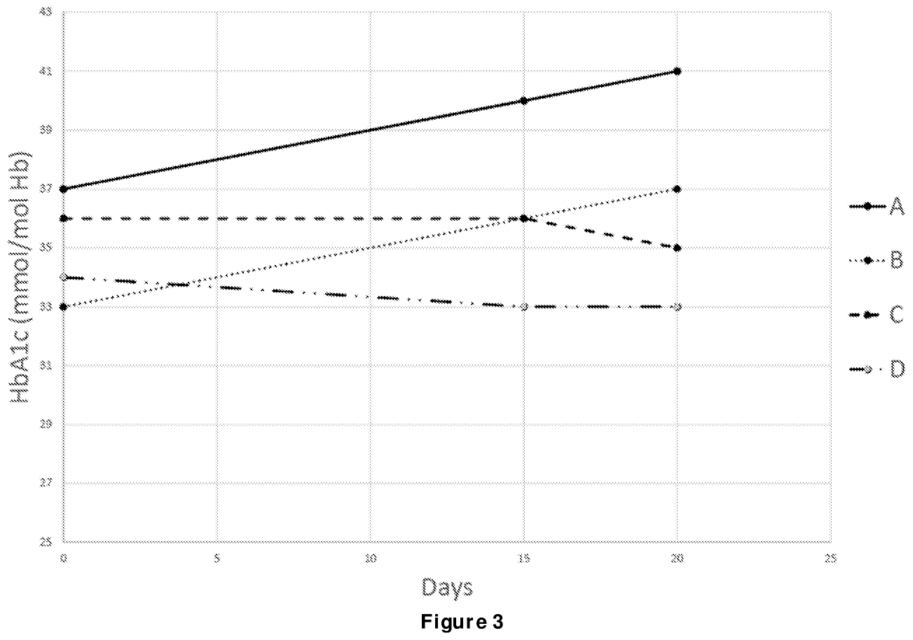

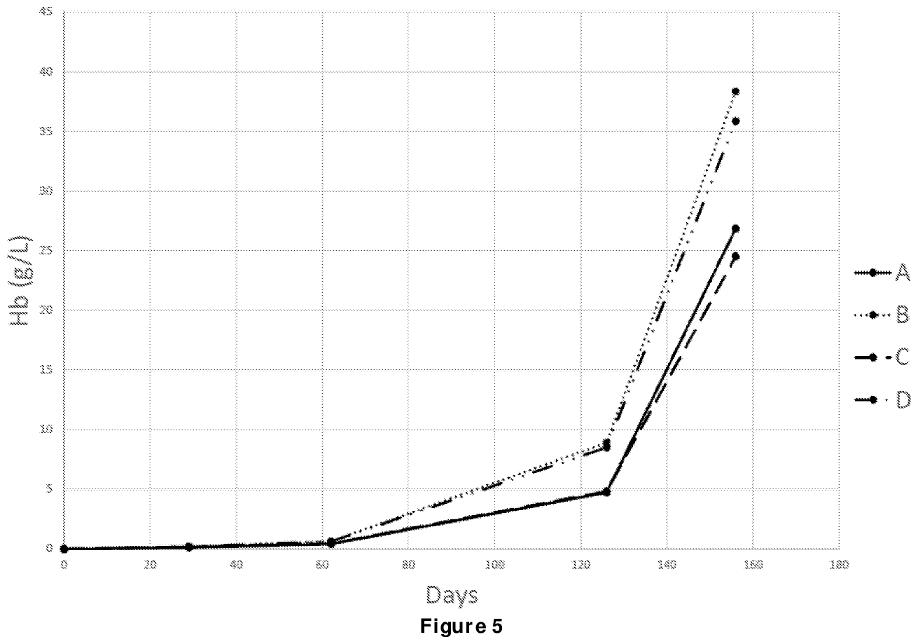

Blood donations processed as outlined above have been prepared and stored at 4° C. for long-term and open stability studies using measurement of HbA1c (see FIGS. 1-4) and supernatant haemolysis (see FIG. 5) on multiple occasions to study sample integrity and degradation. Red cells from two sources were processed and for each two sets of samples produced, one using glucose as the hexose, the other using fructose, to give a total of four sample sets. For long-term stability studies, afresh aliquot of a sample was opened each time, measured and then discarded. In contrast, for the open stability study, one of the sealed aliquots was opened at intervals and measured (i.e. exposing the material to the environment repeatedly over the length of the time period of the study).

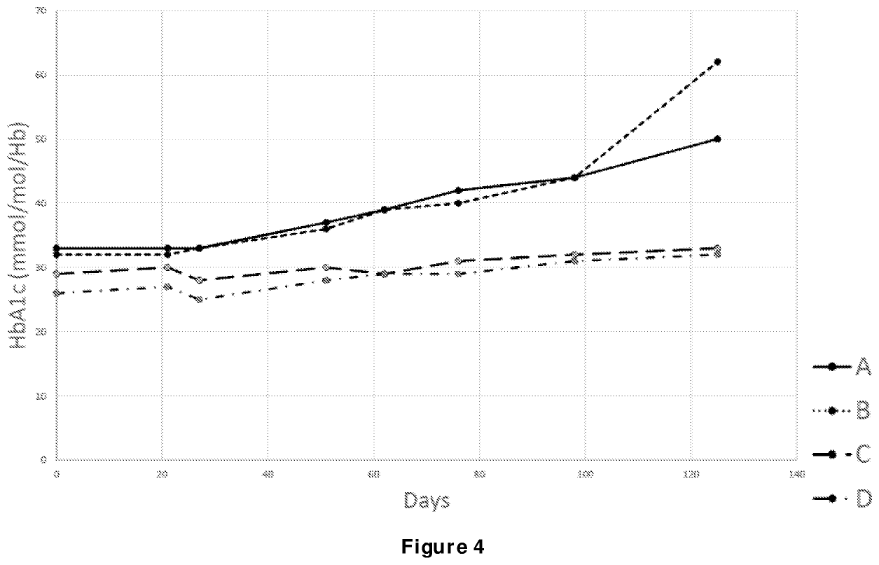

From each sample set (multiple aliquots were available for each set), a sample was used for open stability studies on HbA1c measurement on 3 different platforms (TosohG7, Affinion AS100 and BioRad B100; see FIGS. 1, 2 and 3 respectively) and a series of samples opened and used only once for measurement on the Affinion As100 (long-term stability; see FIG. 4).

Supernatant haemoglobin was also measured periodically on the long-term stability samples by spectrophotometry (for example as described in Fleming, A. P., and Woolf, A. J.: Clin. chim. Acta, 12: 67-74, 1965).

Samples A & B contained Glucose, C & D fructose

Intra-day reproducibility, expressed as a coefficient of variation (CV):

A 1.28%

B 1.08%

C 1.14%

D 1.58%

Inter-day reproducibility

A 5.35%

B 5.52%

C 1.05%

D 2.74%

The coefficient of variation (CV) in clinical pathology=standard deviation/mean×100.

It was observed that the use of glucose/dextrose as the energy source leads to an increase over time in HbA1c (accompanied by an increase in LA1c as seen on the chromatograms from Tosoh and BioRad).

In contrast, use of fructose as the energy source was not accompanied by any significant increase in HbA1c nor of LA1c over time. Hence, fructose is preferred energy source for use in HbA1c control materials.

### EXAMPLE 2

An alternative wash solution and support medium is as follows below

Wash Solution:

Fructose—80 mM

Mannitol—80 mM

K₂H PO4—34.6 mM

KH₂PO₄—5.6 mM

Adenine—2 mM Potassium Citrate—17.9 mM

Support Medium

Fructose—80 mM

Mannitol—80 mM

K₂HPO4—34.6 mM

KH₂PO₄—5.6 mM

Adenine—2 mM

Potassium Citrate—17.9 mM

Bovine serum Albumin—40 g/L

N-Acetylcysteine—0.5 mM

Ascorbic Acid—0.23 mM

Gentamicin—15 mg/L

All publications mentioned in the above specification are herein incorporated by reference. Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

What is claimed:

1. A quality control material comprising:
non-fixed viable red blood cells; and
a support medium comprising:
a buffer;
a polyol;
adenine; and
fructose.

2. A quality control material according to claim 1, wherein the composition further comprises an antimicrobial agent.

3. A quality control material according to claim 2, wherein the antimicrobial agent is selected from the group consisting of: gentamicin, neomycin sulfate, chloramphenicol and a mixture thereof.

4. A quality control material according to claim 1, wherein the composition further comprises an antioxidant selected from the group consisting of: tocopherol acetate, N-acetyl cysteine, ascorbic acid, methyl paraben, esters of para-hydroxybenzoic acid, and a mixture thereof.

5. A quality control material according to claim 1, wherein the polyol is selected from the group consisting of: mannitol, sorbitol, xylitol, glycerol and a mixture thereof.

6. A quality control material according to claim 1, wherein the composition further comprises a protein.

7. A quality control material according to claim 6, wherein the protein comprises a protein selected from the group consisting of: bovine serum albumin, succinylated gelatine and a mixture thereof.

8. A quality control material according to claim 1, wherein the buffer comprises components selected from the group consisting of: citric acid or a salt thereof, phosphate salts and a mixture thereof.

9. A quality control material according to claim 1, wherein the composition further comprises a glucose transport 1 inhibitor.

10. A quality control material according to claim 1, wherein the composition further comprises a stabilizer, an HbA1c enriched haemoglobin solution, a pH adjuster, a protease inhibitor, an analyte of interest or a mixture thereof.

11. A quality control material according to claim 1, wherein the red blood cells are suspended in plasma and the volume/volume ratio of red blood cells in plasma: support medium is from 2:1 to 1:5.

12. A diagnostic test control comprising the quality control material according to claim 1.

13. A diagnostic test control according to claim 12 wherein the diagnostic test control is used in conjunction with a diagnostic test of glycated haemoglobin or haemoglobin A1c.

14. A method for preparing a quality control material according to claim 1, comprising the steps of:
(i) selecting a sample of red blood cells with at least one desired feature from a suitable subject;
(ii) processing the sample to remove white blood cells;
(iii) processing the sample to remove the majority of the plasma;
(iv) optionally washing the sample of step (iii);
(v) optionally re-suspending the sample of step (iv) in plasma;
(vi) admixing the sample of step (v) with a support medium to produce the quality control material.

15. A method for determining the accuracy and reproducibility of an operation of an analytical instrument capable of measuring an analyte of interest comprising:
(a) providing a quality control material according to claim 1 where at least one reference value has been determined;
(b) determining a level of the analyte of interest in the quality control material of (a); and
(c) comparing the level of the analyte of interest obtained in (b) with the at least one reference value; wherein said comparing indicates the accuracy and reproducibility of the operation of the analytical instrument.
